(19) United States
(12) Patent Application Publication
(10) Pub. No.: US 2007/0100388 A1
(43) Pub. Date: May 3, 2007

(54) Implantable medical device providing adaptive neurostimulation therapy for incontinence

(75) Inventor: Martin T. Gerber, Maple Grove, MN (US)

Correspondence Address: MEDTRONIC, INC., 710 MEDTRONIC PARK, MINNEAPOLIS, MN, 55432-9924, US

(73) Assignee: Medtronic, Inc., Minneapolis, MN (US)

(21) Appl. No.: 11/263,170

(22) Filed: Oct. 31, 2005

(51) Int. Cl. A61N 1/32

(52) U.S. Cl. 607/41

(57) ABSTRACT

The invention provides an implantable neurostimulator that includes a pulse generator to supply electrical stimulation through a lead having one or more electrodes, a memory storing at least one fluctuating neurostimulation therapy program, wherein the fluctuating neurostimulation therapy program includes frequency of stimulation, pulse width of stimulation, amplitude of stimulation, particular electrode(s) that is to be utilized, the on and off time, or some combination thereof, and a processor that controls the pulse generator to apply stimulation pulses according to the fluctuating neurostimulation therapy program.

### FIELD OF THE INVENTION

The invention relates to implantable medical devices and, more particularly, devices for delivering neurostimulation therapy for incontinence.

### BACKGROUND

Many people suffer from involuntary urine leakage, i.e., urinary incontinence. Others may suffer from blocked or restricted urine flow. Other urinary disorders include frequent urination, sudden urges to urinate, problems starting a urine stream, painful urination, problems emptying the bladder completely, and recurrent urinary tract infections. A physician uses an urodynamic test to study how a patient stores and releases urine. During the test, the physician obtains urodynamic information based on one or more physiological conditions within the urinary tract.

Different muscles, nerves, organs and conduits within the urinary tract cooperate to collect, store and release urine. A variety of disorders may compromise the urinary tract performance and contribute to incontinence or restricted flow. Many of the disorders may be associated with aging, injury or illness. For example, aging can often result in weakened sphincter muscles, which cause incontinence, or weakened bladder muscles, which prevent complete emptying. Some patients also may suffer from nerve disorders that prevent proper triggering and operation of the bladder or sphincter muscles.

Neurostimulation therapy is applied to alleviate symptoms associated with a variety of pelvic floor disorders including urinary incontinence. An implantable neurostimulator applies electrical stimulation pulses to sacral or pudendal nerves to provide bladder control. Treating physicians have noted that some patients that are treated with neurostimulation therapy will return sometime after implantation and complain of stress urinary incontinence also decreased efficacy over time—some within weeks, other within months or years. The inventor of the present invention has hypothesized that this may be due to fatigue of the muscles with chronic stimulation, accommodation of the muscles (i.e. the muscles become accustomed to the stimulation and don't respond in the same fashion), plasticity of the nervous system, or a combination thereof. This phenomenon is often referred to as nervous system plasticity. The present invention provides a solution to this possible problem.

### SUMMARY

The invention provides an implantable neurostimulator that includes a pulse generator to supply electrical stimulation through a lead having one or more electrodes, a memory storing at least one fluctuating neurostimulation therapy program, wherein the fluctuating neurostimulation therapy program includes frequency of stimulation, pulse width of stimulation, amplitude of stimulation, particular electrode(s) that is to be utilized, the on and off time, or some combination thereof, and a processor that controls the pulse generator to apply stimulation pulses according to the fluctuating neurostimulation therapy program.

The invention also provides a system for providing therapy for at least one pelvic floor disorder that includes an implantable neurostimulator that includes a pulse generator to supply electrical stimulation through a lead having one or more electrodes, a memory storing at least one fluctuating neurostimulation therapy program, wherein the fluctuating neurostimulation therapy program includes frequency of stimulation, pulse width of stimulation, amplitude of stimulation, particular electrode(s) that is to be utilized, the on and off time, or some combination thereof, and a processor that controls the pulse generator to apply stimulation pulses according to the fluctuating neurostimulation therapy program, and at least one implantable lead that includes at least one electrode.

The invention also provides a method for providing therapy for at least one pelvic floor disorder that includes implanting a neurostimulator in a patient, implanting at least one lead having at least one electrode in the patient, and operating the neurostimulator to provide fluctuating neurostimulation therapy to the patient, wherein the fluctuating neurostimulation therapy includes frequency of stimulation, pulse width of stimulation, amplitude of stimulation, particular electrode(s) that is to be utilized, the on and off time, or some combination thereof.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating a neurostimulation system providing adaptive neurostimulation therapy for incontinence.

FIG. 2 is a block diagram illustrating an implantable sensor.

FIG. 3 is a block diagram illustrating an external monitor/programmer.

FIG. 4 is a block diagram illustrating an implantable neurostimulator.

### DETAILED DESCRIPTION OF THE INVENTION

Embodiments of the invention can be utilized to provide therapy and/or affect pelvic floor disorders. Examples of pelvic floor disorders that can be treated using a device and/or method of the invention include, but are not limited to, urinary control disorders, pelvic pain, and fecal control disorders. In one embodiment of the invention, urinary incontinence, fecal incontinence, or some combination thereof are treated using devices and/or methods of the invention.

Embodiments of the invention provide therapy for the various pelvic floor disorders through stimulation of one more nerves of the pelvic floor. Examples of these nerves include, but are not limited to, the sacral nerves, and the pudendal nerves. In one embodiment, the sacral nerves are simulated, in another, the pudendal nerves are stimulated, and in yet another embodiment, both the sacral and pudendal nerves are stimulated.

The invention includes neurostimulation systems that provide fluctuating neurostimulation therapy for incontinence. As used herein, fluctuating neurostimulation therapy can refer to neurostimulation therapy with parameters that, over time, may be less likely to cause nervous system (change throughout) plasticity, muscle fatigue, or some combination thereof. Nervous system plasticity is generally thought of as the ability of the nervous system to adapt to specific stressors and the ability of the system to create alternative nervous system pathways. In the case of neurostimulation therapy, plasticity can be undesirable because the nervous system may learn to accommodate the stimulation thus, minimizing the therapeutic affects which results in a return of disease symptoms. Muscles may begin to respond differently or not at all to the application of electrical stimulation. As used herein, fluctuating neurostimulation therapy can also refer to neurostimulation with parameters that vary over time.

The parameters that can vary include, but are not limited to, frequency, pulse width, amplitude, the particular electrode(s) that is delivering the therapy, on and off time, or some combination thereof. In one embodiment, frequency pulse width, the particular electrode(s) that is delivering the therapy, or some combination thereof may be varied because a patient may be less cognizant of changes in these parameters than amplitude for example. In some embodiments, stimulation parameters, or a range of stimulation parameters will be predefined for a patient. Alternatively, the stimulation parameters or the ranges of stimulation parameters can be gathered after the device has been implanted into the patient. In one embodiment the fluctuations can be based on those predefined, or gathered stimulation parameters or ranges of stimulation parameters. In one embodiment, the stimulation parameters or ranges of stimulation parameters are dictated at least in part by the device itself, the physician, particular efficacious parameters of the patient, or some combination thereof.

Exemplary ranges for neurostimulation stimulation parameters that can be used in the invention and are likely to be effective in treating incontinence, e.g., when applied to the sacral or pudendal nerves, are as follows:

1. Frequency: between about 0.5 Hz and about 500 Hz, in another embodiment between about 10 Hz and about 250 Hz, and in yet another embodiment between approximately 10 Hz and 30 Hz. 2. Amplitude: between about 0.1 volts and about 50 volts, in another embodiment between about 0.5 volts and about 20 volts, and in yet another embodiment between about 1 volt and about 10 volts. 3. Pulse Width: between about 10 microseconds and about 5000 microseconds, in another embodiment between about 100 microseconds and about 1000 microseconds, in yet another embodiment between about 180 microseconds and about 450 microseconds, and in a further embodiment between about 175 microseconds and about 250 microseconds.

In an embodiment where the frequency is varied, the frequency can be varied across the likely range of frequencies. For example, in an embodiment where the frequency can range from about 10 to about 30 Hz, the variability can be from about 10 Hz to about 30 Hz and anywhere in between. In one embodiment where the likely frequency is between about 10 and about 30 Hz, the frequency can be varied between about 10 Hz and about 30 Hz in about 5 Hz increments for example. In another embodiment, the frequency can vary 50% up and/or down from a particular frequency. For example, if about 20 Hz was a commonly used and effective frequency for a patient, a set of fluctuating neurostimulation parameters may range from about 10 Hz to about 30 Hz, or from about 10 Hz to about 20 Hz.

In an embodiment where the pulse width is varied, the pulse width can be varied across the likely range of pulse widths. For example, in an embodiment where the likely pulse width can range from about 175 microseconds to about 250 microseconds, the pulse width can be varied between about 175 microseconds to about 250 microseconds. In one embodiment where the likely pulse width is between about 175 microseconds and about 250 microseconds, the pulse width can be varied between about 175 microseconds and about 250 microseconds in 25 microsecond increments for example. In another embodiment, the pulse width can vary about 50% up and/or down from a particular pulse width. For example, if 175 microseconds was a commonly used and effective pulse width for a patient, a set of fluctuating neurostimulation parameters may range from about 87.5 microseconds to about 262.5 microseconds, or from about 175 microseconds to about 262.5 microseconds.

In an embodiment where amplitude is varied, the amplitude can be varied across the likely range of amplitudes. For example, in an embodiment where the likely amplitude can range from about 1 volt to about 10 volts, the variability can go from about 1 volt to about 10 volts, or anywhere in between. In one embodiment where the likely amplitude is between about 1 volt and about 10 volts, the amplitude can be varied between about 1 volts and 10 volts in 1 volt increments for example. In another embodiment, the amplitude can vary about 50% up and/or down from a particular amplitude. For example, if about 5 volts were commonly used and effective amplitude for a patient, a set of fluctuating neurostimulation parameters may range from about 2.5 Hz to about 7.5 volts, or from 5 volts to about 7.5 volts for example.

In an embodiment where the particular electrodes that are delivering the stimulation are varied, any of the available electrodes on the lead can be utilized. Such an embodiment can be utilized in a lead having more than one electrode. In one embodiment, the lead that is used has four available electrodes. In such an electrode, all four electrodes can be utilized, or less than four, for example three or two, could also be utilized. In one embodiment that utilizes a lead having four electrodes, there may be three of those four electrodes that are more effective for delivering therapy to a particular patient than the fourth electrode. In such an embodiment, those three electrodes that are more effective can be varied to provide fluctuating neurostimulation parameters.

In an embodiment where the on and off time is varied, the stimulation on time can be varied, the stimulation off time can be varied, or some combination thereof. In one embodiment, the amount of time that the stimulation is on is varied, for example, the stimulation can be on for on t₁, the off for an amount of time, and then on for on t₂, and so on, where there either is or is not a relationship between on t₁, on t₂ . . . on t_{∞}. In one embodiment, on t₁, on t₂. . . on t_{∞} are randomly chosen. In another embodiment, the amount of time that the stimulation is off is varied, for example the stimulation is on for an amount of time, on for off t₁, then on, and off for off t₂, and so on, where there either is or is not a relationship between off t₁, off t₂ . . . off t_{∞}. In one embodiment off t₁, off t₂ . . off t_{∞} are randomly chosen. In another embodiment, both on t and off t are randomly chosen. In one embodiment, the on t and off t that are varied can be varied between about 10 seconds to about 10 minutes. In another embodiment, the variation can range from about 30 seconds to about 5 minutes.

Embodiments of the invention vary stimulation parameters such as frequency, pulse width, amplitude, the particular electrodes that are providing the stimulation, the on and off time, or some combination thereof. The variability of the parameters can either be random, non-random (i.e. directed by some pattern), or some combination thereof. In one embodiment, a non-random variability or a variability directed by some pattern includes cycling from one end of a range to another and back again. For example, in an embodiment where the frequency is going to be varied from about 10 Hz to about 30 Hz in 5 Hz increments, the fluctuating neurostimulation parameters could include a repeating cycle of 10 Hz, 15 Hz, 20 Hz, 25 Hz, 30 Hz, 25 Hz, 20 Hz, 15 Hz, 10 Hz, 15 Hz, . . . and so on. Another example of non-random cycling would include going through a range (either from high to low or low to high) and then starting over again and doing the same cycle. An example of this non-random pattern could include a repeating cycle of 10 Hz, 15 Hz, 20 Hz, 25 Hz, 10 Hz, 15 Hz, 20 Hz, 25 Hz, 10 Hz. . . and so on. Another non-random type of variability could include switching between two or more values of the parameter, such as for example 10 Hz, 20 Hz, 10 Hz, 20 Hz, 10 Hz. . . and so on; or 10 Hz, 30 Hz, 10 Hz, 30 Hz, 10 Hz. . . and so on. Any of these types of non-random patterns could be used with any of the stimulation parameters. In an embodiment where the particular electrode that is delivering the therapy is the parameter that is being varied, the location of the electrodes on the lead can be given numerical values based on their location. This location based number can then be used to generate patterns such as those discussed above, and others.

An embodiment where pulse width is randomly varied may be advantageous in that a patient may be less likely to be cognizant of these changes. This is generally in comparison to random changes in some of the other stimulation parameters, such as frequency and amplitude where random changes may be noted by the patient as producing noticeable effects on the patient.

In one embodiment, two parameters are varied. For example, pulse width and frequency can both be varied. They can both be randomly varied, both non-randomly varied, or one randomly varied and the other non-randomly varied. In embodiments where two parameters are varied, they can either be changed simultaneously or at different times. For example, in one embodiment, the frequency (for example) can be changed at t₁and then the pulse width (for example) can be changed at time t₂. In another embodiment, the frequency and the pulse width (for example) can both be changed at time t₁ and then both changed again at time t₂.

In one embodiment the power that is delivered to the patient can be randomly or non-randomly varied. The power is a function of both the frequency and the pulse width. In embodiments where the ultimate power is varied, the changes to frequency and pulse width are generally both made at the same time, e.g. at time t1 the frequency and pulse width have values of x₁ and y₁; and at time t₂, the frequency and pulse width have values of x₂ and y₂. One example includes a random variation in power which could be accomplished by a random variation in one of either frequency or pulse width, which would then dictate the other. For example, the power can be randomly chosen and the frequency can be randomly chosen. The pulse width would then be dictated by the randomly chosen power and frequency values. Alternatively, a random variation in power could be accomplished by a non-random cycling of frequency (for example) which would then dictate the pulse width.

An embodiment also includes a non-random variation in power which could be accomplished by a random variation in frequency (for example) which would then dictate the pulse width. Alternatively, a non-random variation in power could also be accomplished by a non-random cycling of frequency (for example) which would then dictate a non-random cycling of pulse width. One of skill in the art, having read this specification, will understand that any combination of random and non-random variation of power, frequency, and pulse width that was not explicitly discussed herein is also included in the invention.

Another factor to be considered is when and how often the variation in stimulation parameters is carried out. Generally, the variation can occur at any period, i.e. on any time frame, for example including, but not limited to, second to second, minute to minute, hour to hour, day to day, etc. In one embodiment, a variation in stimulation parameter occurs on a daily basis. In another embodiment a variation in stimulation parameters occurs twice a day, for example at a time that is set to coordinate at least somewhat with expected waking and sleeping times. In one embodiment, the time at which the variation is accomplished is independent of other factors (such as expected sleeping and waking times) and can change as time goes on.

The use of fluctuating neurostimulation parameters can begin immediately upon implantation of the neurostimulator or can begin at some later time after the implantation. In one embodiment, the fluctuating neurostimulation parameters replace non-fluctuating neurostimulation parameters once symptoms of muscle plasticity, such as stress urinary incontinence are recognized by the patient or decrease in efficacy, the doctor, or both. In another embodiment, the fluctuating neurostimulation parameters replace non-fluctuating neurostimulation parameters once symptoms of muscle plasticity are detected by one or more sensor that is part of the neurostimulation system of one embodiment of the invention. In another embodiment, the use of fluctuating neurostimulation parameters can be discontinued once the. symptoms of muscle plasticity are no longer detected by the one or more sensor that is part of the neurostimulation system of one embodiment of the invention.

In one embodiment, the patient is able to begin the use of fluctuating neurostimulation parameters without further intervention by a physician. In another embodiment, the patient must obtain physician approval of this change. Of course, the patient can schedule an appointment to see the physician to gain approval of this change (if necessary), or alternatively methods and systems for physician approval similar to those described in commonly assigned U.S. patent application Ser. No. 11/1368 16,963 entitled “Implantable Medical Device Providing Adaptive Neurostimulation Therapy for Incontinence”, the disclosure of which is incorporated herein by reference, can be utilized.

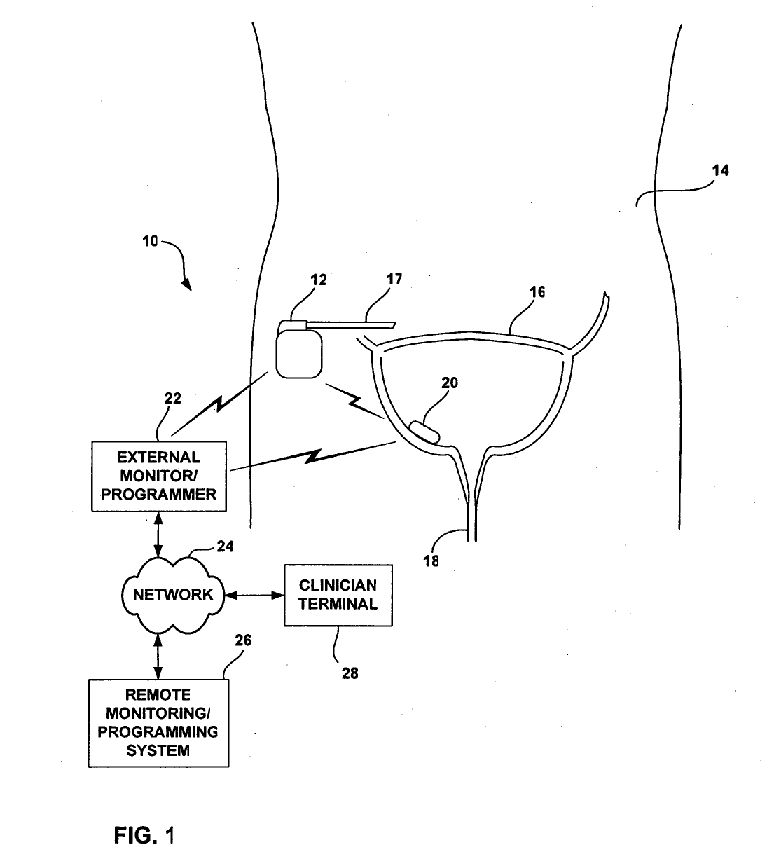

As shown in FIG. 1, system 10 includes an implantable neurostimulator 12. Neurostimulator 12 is implanted within patient 14 to deliver neurostimulation therapy for control of the finction of bladder 16 for example. Neurostimulator 12 may include at least one lead 17 carrying one or more electrodes for delivery of neurostimulation pulses to sacral nerves within the pelvic floor of patient 14. One embodiment can also include one or more implanted urodynamic sensors 20 within bladder 16 to sense physiological conditions such as flow, pressure, contractile force and the like. Sensor 20 may be implanted within bladder 16, urethra 18 or elsewhere within the body of patient 14. Also, in some embodiments, multiple sensors 20 may be implanted within patient 14.

In embodiments that include sensor 20, neurostimulator 12 may receive information from sensor 20 via wireless telemetry. In addition, an external monitor/programmer 22 may receive information from neurostimulator 12 and/or sensor 20 by wireless telemetry. In alternative embodiments, sensor 20 may be integrated within the housing of neurostimulator 12 or coupled to the neurostimulator 12 via one or more leads. External monitor/programmer 22 also may transmit information to neurostimulator 12. For example, external monitor/programmer 22 may take the form of a patient programmer that receives information from patient 14 as user input provided via a user interface.

Sensor 20, or multiple sensors, may provide a variety of information indicative of the level of efficacy achieved by the neurostimulation therapy delivered by neurostimulator 12. The information may be any information relating to the function of the bladder 16, or any other segment of the patient's urinary tract, in storing releasing and passing urine. For example, sensor 20 may monitor parameters such as bladder pressure, bladder contractile force, urinary sphincter pressure, urine flow rate, urine flow pressure, voiding amount, and the like. Other examples of sensed information include urine flow velocity, urine or bladder temperature, impedance, urinary pH, or chemical constituency of the urine. Any of such information may reveal the effect of the neurostimulation therapy on the physiological function of bladder 16, urethra 18 or the urinary sphincter. Such information may also reveal changes in efficacy that may be a result of plasticity. Such sensor readings could then be utilized to indicate a change, or the beginning of using fluctuating neurostimulation therapy.

In still other embodiments, one or more sensors 20 may be implanted within patient 14 to sense a physiological state of the patient. For example, a sensor may be deployed to sense cardiac activity, respiratory activity, electromyographic activity, or the like, as an indication of patient activity level. Such activity level information, in conjunction with other information, may be useful in determining adjustments to stimulation parameters. Other types of sensors 20 also may detect a posture or activity level of the patient. For example, an accelerometer may detect an elevated activity level, e.g., during exercise, while other sensors may detect whether the patient is sitting, standing or lying down. In addition, some of the information obtained by such sensors, such as respiration activity, may be analyzed to determine, e.g., whether the patient is sleeping.

Sensor 20 may be chronically implanted within patient 14 for use over an extended period of time. In this case, sensor 20 carries sufficient battery resources, a rechargeable battery, or an inductive power interface that permits extended operation. Sensor 20 may be implanted by minimally invasive, endoscopic techniques for an extended period of time or a limited period of time to capture information useful in analyzing and adjusting the stimulation parameters. In other words, sensor 20 may be chronically implanted to support ongoing parameter adjustments over an extended course of therapy spanning several months or years, or purposefully implanted for a short period of time to support a one-time parameter adjustment or a small number of adjustments over a relatively short period of time, such as several hours, days or weeks.

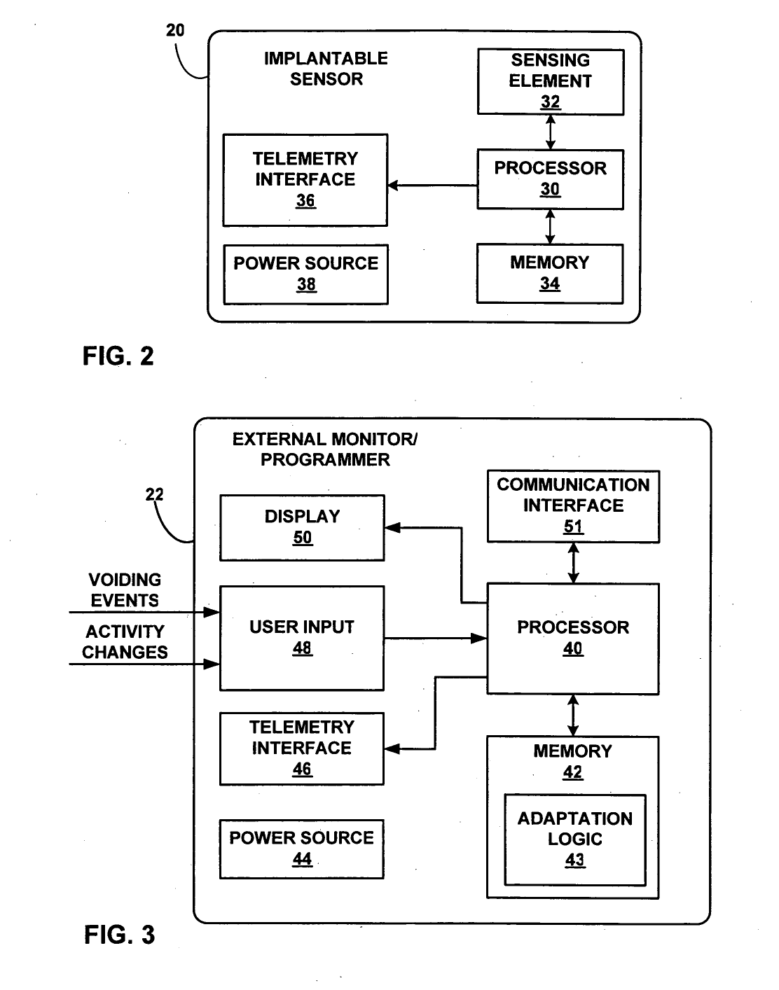

FIG. 2 is a functional block diagram illustrating implantable sensor 20 of FIG. 1. In the example of FIG. 2, sensor 20 includes a sensor processor 30, a sensing element 32, memory 34, wireless telemetry interface 36, and a power source 38. Sensor 20 also may include an internal clock to track date and time of voiding events. Sensor 20 may have a capsule-like shape, and may be placed within bladder 14 or urethra 18 by endoscopic introduction via the urethra, or by hypodermic injection using a hypodermic needle. Alternatively, sensor 20 may be surgically implanted. In the case of minimally invasive endoscopic introduction, sensor 20 may be constructed in a manner similar to the sensors described in U.S. patent application Ser. No. 10/978,233, to Martin Gerber, filed Oct. 29, 2004, and entitled “Wireless Urinary Voiding Diary System,” which claims the benefit of U.S. provisional application no. 60/589,442, filed Jul. 20, 2004; or U.S. patent application Ser. No. 10/833,776, to Mark Christopherson and Warren Starkebaum, filed Apr. 28, 2004, entitled “Implantable Urinary Tract Monitor,” the entire content of each of which is incorporated herein by reference.

Power source 38 may take the form of a small battery. An external source of inductively coupled power may be used, in some embodiments, to power some features of monitor 20, or to recharge the battery. For example, sensor 20 may include an inductive power interface for transcutaneous inductive power transfer to power higher energy functions such as telemetry. However, sensor 20 typically will include a small battery cell within the sensor housing. Alternatively, sensor 20 may include an inductive power interface in lieu of a battery.

Telemetry interface 36 permits wireless communication with external monitor/programmer 22, remote monitoring/programming system 26, or neurostimulator 12 for wireless transmission of information obtained by sensor 20, as well as wireless reception of activation triggers that direct sensor 20 to collect physiological information or transmit stored information. As a further alternative, triggered activation may be applied by patient 14 in the form of a magnet swiped in proximity to sensor 20, in which case the monitor will include appropriate sensing circuitry to detect the magnet.

Sensor processor 30 controls telemetry interface 36 and handles processing and storage of information obtained by sensing element 32. Sensor processor 30 controls operation of sensor 20 and may include one or more microprocessors, digital signal processors (DSPs), application-specific integrated circuits (ASICs), field-programmable gate arrays (FPGAs), or other equivalent logic circuitry. Memory 34 may include any magnetic, electronic, or optical media, such as random access memory (RAM), read-only memory (ROM), electronically-erasable programmable ROM (EEPROM), flash memory, or the like, or a combination thereof. Memory 34 may store program instructions that, when executed by sensor processor 30, cause the controller to perform the functions ascribed to it herein. For example, memory 34 may store instructions for sensor processor 30 to execute in support of control of wireless telemetry interface 36 and control of, and processing of information obtained by, sensing element 32. Memory 34 may include separate memories for storage of instructions and urodynamic information.

Telemetry interface 36 may include a wireless radio frequency (RF) transmitter and receiver to permit bi-directional communication between sensor 20, neurostimulator 12, external monitor/programmer 22, remote monitoring/programming system 26, or some combination thereof. In this manner, external monitor/programmer 22 may transmit commands to sensor 20 for collection of information or collection of information stored in memory 34, and receive status and operational information from the sensor 20. Telemetry interface 36 includes an antenna, which may take a variety of forms. For example, the antenna may be formed by a conductive coil or wire embedded in a housing associated with sensor 20. Alternatively, the antenna may be mounted on a circuit board carrying other components of sensor 20, or take the form of a circuit trace on the circuit board.

Battery power source 38 may take the form of a battery and power generation circuitry. In some embodiments, sensor 20 may be used for a few days or weeks, and therefore may not require substantial battery resources. Accordingly, the battery within battery power source 38 may be very small in some cases. An example of a suitable battery is the Energizer 337 silver oxide cell, available from the Eveready Battery Company, of St. Louis, Mo., USA. The Energizer 337 battery is disc-shaped, and has a diameter of 4.88 mm and thickness of 1.65 mm. Another example battery is the QL003I 3 milliamp cylindrical battery from Quallion, LLC, of Sylmar, Calif., USA, which has a diameter of approximately 2.9 mm and a length of approximately 13.0 mm.

In further embodiments, battery power source 38 may be rechargeable via electromagnetic induction or ultrasonic energy transmission, and includes an appropriate circuit for recovering transcutaneously received energy. For example, battery power source 38 may include a secondary coil and a rectifier circuit for inductive energy transfer. In still other embodiments, battery power source 38 may not include any storage element, and sensor 20 may be fully powered via transcutaneous inductive energy transfer, which may be provided by external receiver 14. In either case, sensor 20 may be constructed for short-term or long-term operation.

Sensing element 32 may be selected for any of a variety of urodynamic testing applications, and may include appropriate signal processing circuitry such as amplifier, filter, driver, and analog-to-digital conversion circuitry for presentation of sensed information to sensor processor 30. For urodynamic testing, sensing element 32 may take the form of a pressure, flow, velocity, volume, temperature, impedance, or contractile force sensor. For pressure measurements, for example, sensing element 32 may include one or more diaphragm sensors, strain gauge sensors, capacitive sensors, piezoelectric sensors, or other sensors used in conventional catheter-based urodynamic testing to sense pressure. As a further example, for bladder emptying, sensing element 32 may include a conductive sensor to sense the presence of urine within the lower region of the bladder 16.

For flow measurements, sensing element 32 may comprise a pulsed Doppler ultrasonic sensor, or a laser Doppler flow sensor. Doppler shifting of the frequency of the reflected energy indicates the velocity of the fluid flow passing over a surface of sensing element 32. Consequently, in some embodiments, sensor 20 may include circuitry, such as a quadrature phase detector, in order to enable the monitor to distinguish the direction of the flow of fluid in addition to its velocity.

As a further example, sensing element 32 may include any one or more thermal-convection velocity sensors. A thermal-convection velocity sensor may include a heating element upstream of a thermistor to heat urine within the urethra 18 such that flow rate may be measured according to the temperature of the heated fluid when it arrives at the thermistor. In other embodiments, flow rate may be determined from the output of a concentration or temperature sensor using Fick's techniques.

In some embodiments, sensing element 32 may include multiple sensors of a given type, as well as multiple types of sensors, e.g., pressure, flow, bladder emptying, or the like. Accordingly, the information obtained by sensor 20 may then include different types of physiological parameters associated with a voiding event. Alternatively, multiple sensors 20 may be deployed within bladder 16 or urethra 18. In this case, each sensor 20 may be configured with a different type or set of sensing elements 32 to collect a variety of different undynamic parameters during a voiding event.

In some other embodiments, sensing element 32 may be chosen to sense a physiological state, such as an activity type, activity level, or posture of the patient 14. For example, sensing element 32 can include an accelerometer to detect an elevated activity level, or a decreased activity level.

FIG. 3 is a functional block diagram illustrating external monitor/programmer 22. In the example of FIG. 3, external monitor/programmer 22 includes a processor 40, memory 42, power source 44, telemetry interface 46, user input device 48 and display 50. User input device 48 may take the form of a set of buttons, a keypad, a touchscreen, soft keys on a display, or other input media. Display 50 may be a liquid crystal display (LCD), plasma display, or the like, which conveys status and operational information to the patient 14, and aids the patient in entry of information into external monitor/programmer 22 if that is utilized in the particular embodiment of the invention.

Memory 42 stores instructions for execution by processor 40, as well as a set of fluctuation logic 43, which can be used to determine and/or utilize the fluctuation neurostimulation parameters. In one embodiment, the fluctuation neurostimulation parameters are predetermined and stored in the memory 42 instead of storing fluctuation logic to determine those parameters. In addition, memory 42 may store information received from sensor 20, neurostimulator 12, and patient 14. Memory 42 may include separate memories for storage of instructions and information received from sensor 20, neurostimulator 12 or patient 14. Processor 40 may be constructed in a variety of ways, as described above with respect to sensor processor 30 of FIG. 2, including as one or more microprocessors, an ASIC, an FPGA, or a combination thereof. It should also be understood and appreciated by one of skill in the art that the functions of the processor 40 as described above with respect to the fluctuation logic could be undertaken by a similar processor associated with the neurostimulator 12, the sensor 20, the remote monitoring/programming system 26, or some combination thereof.

In one embodiment, processor 40 controls telemetry interface 46 to obtain urodynamic information from sensor 20 (if present), neurostimulator 12, or some combination thereof. Processor 40 also may control telemetry interface 46 to receive information from sensor 20 or neurostimulator 12 on a substantially continuous basis, at periodic intervals, or only upon receipt of an activation command.

Wireless telemetry may be accomplished by radio frequency (RF) communication or proximal inductive interaction of external monitor/programmer 22 with sensor 20 or neurostimulator 12. Alternatively, telemetry interfaces 36, 46 may be configured for sensor 20 and external monitor/programmer 22 to support radio frequency (RF) communication with a sufficiently strong signal such that proximate interaction is not required. In addition to an RF or inductive telemetry interface 46, external monitor/programmer 22 may include a wired or wireless interface 51 for communication with other external devices, e.g., either directly or via network 24.

External monitor/programmer 22 may take the form of a portable, handheld device, like a pager, cell phone, or patient programmer that can be carried by patient 14. External monitor/programmer 22 may include an internal antenna, an external antenna protruding from the device housing, or an external antenna that extends from the device housing on a cable and is attached to the body of patient 14 at a location proximate to the location of neurostimulator 12 or sensor 20 to improve wireless communication reliability. Also, in some embodiments, external monitor/programmer 22 also may receive operational or status information from neurostimulator 12 or sensor 20, and may be configured to actively configure and interrogate the neurostimulator 12 or sensor 20 to receive the information.

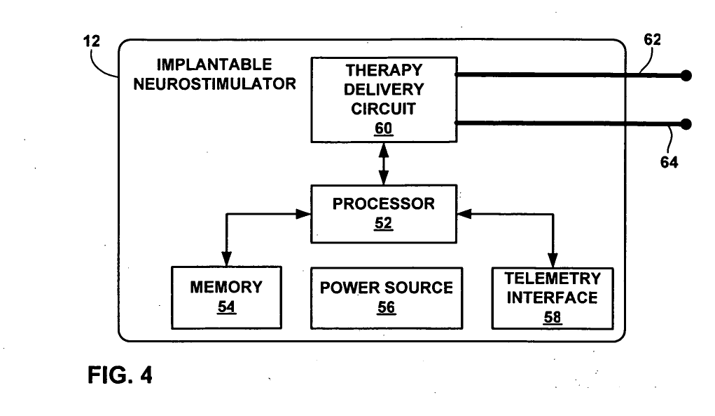

FIG. 4 is a block diagram illustrating neurostimulator 12. As shown in FIG. 4, neurostimulator 12 can include a processor 52, memory 54, power source 56, telemetry interface 58, and therapy delivery circuit 60. Memory 54 stores one or more neurostimulation programs and/or one or more fluctuation neurostimulation programs that specify neurostimulation parameters for stimulation pulses delivered by therapy delivery circuit 60. The parameters may be adjusted automatically or upon clinician approval by external monitor/programmer 22, which downloads or inputs new programs, new parameters or stimulation parameter adjustments to neurostimulator 12.

In general, the stimulation parameters are selected to have values effective in controlling or managing symptoms of urinary incontinence, such as involuntary leakage. The particular parameters were discussed above, and will not be reiterated here.

Therapy delivery circuit 60 drives one or more leads. In the example of FIG. 4, therapy delivery circuit 60 drives electrodes carried by a pair of leads 62, 64. Leads 62, 64 extend from the housing of neurostimulator 12, and have a distal end that extends to target nerve sites within the pelvic floor, such as sacral or pudendal nerve sites. Each lead 62, 64 may carry one of more electrodes, and may be configured as an axial lead with ring electrodes or a paddle lead with electrode pads arranged in a two-dimensional array. The electrodes may operate in a bipolar or multi-polar configuration with other electrodes, or may operate in a unipolar configuration referenced to an electrode carried by the device housing or “can” of neurostimulator 12.

Power source 56 may be a battery, either rechargeable or non-rechargeable. In the case of a rechargeable battery, power source 56 may include an inductive power interface for recharging. In other embodiments, power source 56 may be powered entirely by inductive power transfer from an external power source. Telemetry interface 58 may be constructed and function in a manner similar to telemetry interface 36 of implantable sensor 20 of FIG. 2. Processor 52 may be constructed in a variety of ways, as described above with respect to sensor processor 30 of FIG. 2, including as one or more microprocessors, an ASIC, an FPGA, or a combination thereof.

One embodiment of the invention may also utilize a remote monitoring/programming system. An example of such a system was described in commonly assigned U.S. patent application Ser. No. 11/116,963 entitled “Implantable Medical Device Providing Adaptive Neurostimulation Therapy for Incontinence”, the disclosure of which is incorporated herein by reference.

Many embodiments of the invention have been described. Various embodiments may be adapted to provide adaptive neurostimulation for other pelvic floor disorder such as fecal incontinence, sexual dysfunction, pelvic pain, cystitis, or the like. Accordingly, while the invention has been described in the context of urinary incontinence for purposes of illustration, it is not so limited.

Many embodiments of the invention have been described. These and other embodiments are within the scope of the following claims.

1. An implantable neurostimulator comprising:
a pulse generator to supply electrical stimulation through a lead having one or more electrodes;
a memory storing at least one fluctuating neurostimulation therapy program, wherein the fluctuating neurostimulation therapy program comprises frequency of stimulation, pulse width of stimulation, amplitude of stimulation, particular electrode(s) that is to be utilized, the on and off time, or some combination thereof; and
a processor that controls the pulse generator to apply stimulation pulses according to the fluctuating neurostimulation therapy program.

2. The implantable neurostimulator according to claim 1, wherein the at least one fluctuating neurostimulation therapy program varies based on the frequency of stimulation.

3. The implantable neurostimulator according to claim 2, wherein the frequency of stimulation varies from about 10 Hz to about 30 Hz.

4. The implantable neurostimulator according to claim 2, wherein the frequency of stimulation is randomly varied.

5. The implantable neurostimulator according to claim 2, wherein the frequency of stimulation is non-randomly varied.

6. The implantable neurostimulator according to claim 1, wherein the at least one fluctuating neurostimulation therapy program varies based on the pulse width of stimulation.

7. The implantable neurostimulator according to claim 6, wherein the pulse width varies from about 175 microseconds to about 250 microseconds.

8. The implantable neurostimulator according to claim 6, wherein the pulse width of stimulation is randomly varied.

9. The implantable neurostimulator according to claim 6, wherein the pulse width of stimulation is non-randomly varied.

10. The implantable neurostimulator according to claim 1, wherein the at least one fluctuating neurostimulation therapy program varies based on the on and off time.

11. The implantable neurostimulator according to claim 10, wherein the on time, the off time, or both the on time and the off time are varied between about 30 seconds and about 5 minutes.

12. The implantable neurostimulator according to claim 11, wherein the on time, the off time, or both the on time and the off time are randomly varied.

13. The implantable neurostimulator according to claim 1 further comprising at least one sensor that monitors at least one parameter that could reveal changes in the efficacy of the neurostimulation therapy.

14. The implantable neurostimulator according to claim 13, wherein the fluctuating neurostimulation therapy program is utilized as a result of the at least one sensor monitors a change in a parameter.

15. A method for providing therapy for at least one pelvic floor disorder comprising:
implanting a neurostimulator in a patient;
implanting at least one lead having at least one electrode in the patient;
operating the neurostimulator to provide fluctuating neurostimulation therapy to the patient, wherein the fluctuating neurostimulation therapy comprises frequency of stimulation, pulse width of stimulation, amplitude of stimulation, particular electrode(s) that is to be utilized, the on and off time, or some combination thereof.

16. The method according to claim 15 further comprising the step of monitoring the efficacy of the therapy before operating the neurostimulator to provide fluctuating neurostimulation therapy.

17. The method according to claim 16, wherein the step of monitoring the efficacy of the therapy comprises patient monitoring, physician monitoring, monitoring via a sensor, or some combination thereof.

18. The method of claim 15, wherein the pelvic floor disorder is incontinence, a gastric mobility disorder, pain relief, sexual dysfunction, interstitial cystitis, or some combination thereof.

19. An implantable neurostimulator configured to perform the method of claim 15.

20. A system for providing therapy for at least one pelvic floor disorder comprising:
an implantable neurostimulator comprising:
a pulse generator to supply electrical stimulation through a lead having one or more electrodes;
a memory storing at least one fluctuating neurostimulation therapy program, wherein the fluctuating neurostimulation therapy program comprises frequency of stimulation, pulse width of stimulation, amplitude of stimulation, particular electrode(s) that is to be utilized, the on and off time, or some combination thereof; and
a processor that controls the pulse generator to apply stimulation pulses
according to the fluctuating neurostimulation therapy program; and at least one implantable lead that comprises at least one electrode.

21. The system according to claim 20, wherein the at least one fluctuating neurostimulation therapy program varies based on the frequency of stimulation.

22. The system according to claim 21, wherein the frequency of stimulation varies from about 10 Hz to about 30 Hz.

23. The system according to claim 21, wherein the frequency of stimulation is randomly varied.

24. The system according to claim 21, wherein the frequency of stimulation is non-randomly varied.

25. The system according to claim 20, wherein the at least one fluctuating neurostimulation therapy program varies based on the pulse width of stimulation.

26. The system according to claim 25, wherein the pulse width varies from about 175 microseconds to about 250 microseconds.

27. The system according to claim 25, wherein the pulse width of stimulation is randomly varied.

28. The system according to claim 25, wherein the pulse width of stimulation is non-randomly varied.

29. The system according to claim 20, wherein the at least one fluctuating neurostimulation therapy program varies based on the on and off time.

30. The system according to claim 29, wherein the on time, the off time, or both the on time and the off time are varied between about 30 seconds and about 5 minutes.

31. The system according to claim 30, wherein the on time, the off time, or both the on time and the off time are randomly varied.

32. The system according to claim 20 further comprising at least one sensor that monitors at least one parameter that could reveal changes in the efficacy of the neurostimulation therapy.

33. The system according to claim 32, wherein the fluctuating neurostimulation therapy program is utilized as a result of the at least one sensor monitors a change in a parameter.
